(19) United States
(12) Patent Application Publication
(10) Pub. No.: US 2005/0240837 A1
(43) Pub. Date: Oct. 27, 2005

(54) Control unit and method of operating a control unit

(75) Inventors: Laurent Dubois, Caen (FR); Philippe Raoul, Fresne LaMere (FR); Gerhard Mersch, Uetze (DE)

Correspondence Address: CARLSON, GASKEY & OLDS, P.C., 400 WEST MAPLE ROAD, SUITE 350, BIRMINGHAM, MI, 48009, US

(73) Assignee: ArvinMeritor GmbH

(21) Appl. No.: 11/115,562

(22) Filed: Apr. 27, 2005

(30) Foreign Application Priority Data
Apr. 27, 2004 (DE) 10 2004 020 564.7

(51) Int. Cl. A61K 51/00; A61M 36/14; G01R 31/28

(52) U.S. Cl. 714/712

(57) ABSTRACT

A method of operating a control unit (CU) begins by performing a power on reset. If the CU receives a start-up message within a predefined time period after the reset, then the CU enters a diagnostic mode in which a diagnostic communication can be established with a diagnostic unit. If no start-up message is received within the predefined time period, the CU enters a normal mode of operation. The CU includes a memory designated to store data unique for a respective vehicle type, a first communication module allowing communication via a bus system under a first protocol for normal operation of the CU, and a second communication module allowing communication via the bus system under a second protocol for accessing the memory.

### RELATED APPLICATIONS

The application claims priority to German Application No. 10 2004 020 564.7, which was filed on Apr. 27, 2004.

### BACKGROUND OF THE INVENTION

The invention relates to a control unit and a method of operating the control unit, which is particularly suitable for automotive applications.

A control unit (CU) controls operation of an electronic, electric or electromechanical system. In an attempt to reduce the number of different control units that need to be developed and manufactured, it is known to use a “standardized” control unit for a specific application. This standardized control unit has a memory that allows storage of unique or specific data by which the control unit is adapted to corresponding specific operating conditions or parameters, e.g. to a specific vehicle type in which the control unit is used.

Using an erasable memory for storing the unique or specific data allows subsequent modification of the specific data if, during series production of the vehicle, it is determined that the data should be modified in order to adapt the operation of the control unit and the respective system to revised parameters.

One problem associated with the unique data being modifiable or erasable is that unauthorized modification is possible. Unauthorized modification could result in either amending the parameters that are essential for performing the respective function or even switching off this function. This problem is described with reference to a door control unit as a typical example.

A door control unit (DCU) controls a power window drive by which a window can be displaced from a closed position towards an open position, and vice-versa. In modern vehicles, power window drives are not directly connected to switches that are actuated by a vehicle occupant for opening or closing a vehicle window. Rather, the power window drives (and possibly the switches) are connected to a bus system by which these components communicate with each other. An example of a bus system is a controller area network (CAN) bus.

In addition to simply opening and closing the respective window, the DCU is increasingly used to provide for additional functions that are related to the respective window and/or the respective vehicle door. An example of such a function is an anti-squeeze function, which prevents parts of a vehicle occupant from being squeezed between a window frame and the respective window as the window is driven towards the closed position. The anti-squeeze function can be implemented on the basis of a maximum allowable drive force (or maximum allowable current) that is applied to the window (or a motor of the power window drive) during closing of the window. Exceeding the maximum allowable drive force (or the maximum allowable current) is interpreted as an inadmissible condition that possibly was caused by the window encountering an obstacle in its path, for example a part of the body of a vehicle occupant. If an obstacle is encountered, closing of the window is stopped immediately.

The details of such an anti-squeeze function depends from certain parameters that are specific for each vehicle type. One parameter is friction that occurs when the window is displaced in the window frame. Another parameter is the maximum allowable drive force that is considered by respective vehicle manufactures as being permissible. These parameters are stored in a memory of the DCU. This allows largely standardized DCUs to be manufactured, which are then customized for the respective vehicle type by storing data unique for the vehicle type in the memory. This further allows subsequent modification of the unique data if, during series production of the vehicle, it is determined that the data should be modified in order to adapt the operation of the DCU and the power window drive to revised parameters.

Should the unique data be modified or erased by unauthorized or unintentional access, such access could result in either amending the parameters that are essential for performing the respective function, or even switching off of this function. In the above example of a DCU, switching the anti-squeeze function off is clearly undesirable.

In the prior art, attempts were made to allow access to the memory only if a certain diagnostic code was sent via the bus system. However, there remains a risk of unauthorized or unintentional access to the memory for two reasons. First, there remains a risk that a message sent via the bus system and intended for a component different from the respective CU is falsely interpreted by the respective CU as diagnostic code. This is due to the fact that many different components communicate via the bus system, which results in a plurality of different messages being sent via the bus system. All of these messages are communicated to, i.e., “overheard” by, the CU. Second, there remains a risk that the specific parameters (such as the anti-squeeze function in the above example) are unintentionally modified when accessing the CU for service purposes. Such access can be made for reading a failure memory in which data indicating malfunction or unusual operation conditions are stored, or for updating a program that controls operation of a system associated with the CU.

### SUMMARY OF THE INVENTION

The object of the invention is to reliably allow access to specific data of a control unit (CU) to authorized personnel only, and to prevent any unintentional or unauthorized access to data that is considered as relevant for correct operation of the CU and systems that are controlled by the CU.

This is accomplished by providing a method of operating a control unit (CU) that includes the following steps. First, a power on reset is performed. If the CU receives a start-up message within a predefined time period after the reset, then the CU enters a diagnostic mode in which a diagnostic communication can be established with a diagnostic unit. + If no start-up message is received within the predefined time period, the CU enters a normal mode of operation.

Two different modes of operation are used. In the normal mode of operation, the CU operates in a conventional manner. In this mode of operation, no diagnostic communication can be established, and no access to unique data is possible. In a diagnostic mode of operation, access to the unique data is possible. Unintentionally entering the diagnostic mode of operation is prevented by two criteria. First, a start-up message should be received after a power on reset. This prevents the CU from entering a diagnostic mode during normal operation as a power on reset (which is defined by interrupting power supply for a period of time, e.g., a few seconds) is only performed on rare occasions. Second, the start-up message should be received within a predefined (typically very short) time period after the power on reset. This further limits the risk of unintentionally entering the diagnostic mode. Additional security against unintentional or unauthorized access to the unique data is achieved by using a communication protocol for the diagnostic mode that is different from a communication protocol used in the normal mode of operation. As the communication protocol for the diagnostic mode is not used for other purposes, vehicle manufacturers and repair or service shops need not have access to this communication protocol, thereby preventing access to the unique data.

The invention also provides a control unit (CU) having a memory designated to store data unique for a respective vehicle type. The CU includes a first communication module allowing communication via a bus system under a first protocol for normal operation of the CU, and a second communication module allowing communication via the bus system under a second protocol for accessing the memory. Regarding the advantages achieved with this CU, reference is made to the above comments.

These and other features of the present invention can be best understood from the following specification and drawings, the following of which is a brief description.

### BRIEF DESCRIPTION OF THE DRAWINGS

FIG. 1 schematically shows a control unit (CU) designed according to the subject invention.

FIG. 2 is a schematic view of a vehicle provided with the CU as shown in FIG. 1.

FIG. 3 is a block diagram showing steps of the method incorporating the subject invention.

### DETAILED DESCRIPTION OF THE PREFERRED EMBODIMENT

The invention is hereinafter described with reference to a door control unit (DCU) 10. However, it should be understood that the invention could also be utilized with other kinds of control units.

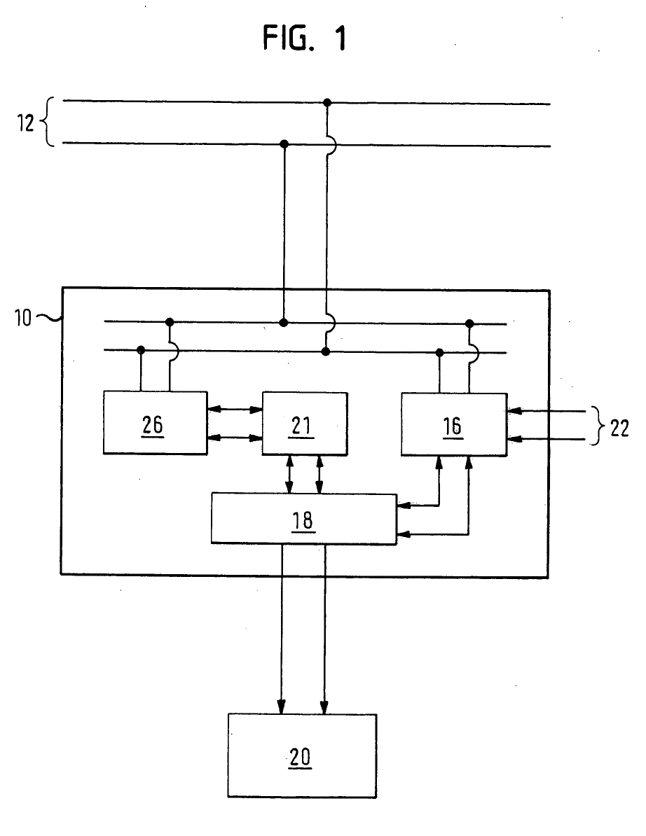

FIG. 1 schematically shows a DCU 10 that is used in vehicles for controlling and operating a door function, such as a power window drive for example. Other vehicle functions could also be controlled by such a control unit. For example, control of a central closing (CC) system for vehicle doors, or control over vehicle interior lighting systems could also be accomplished with such a control unit.

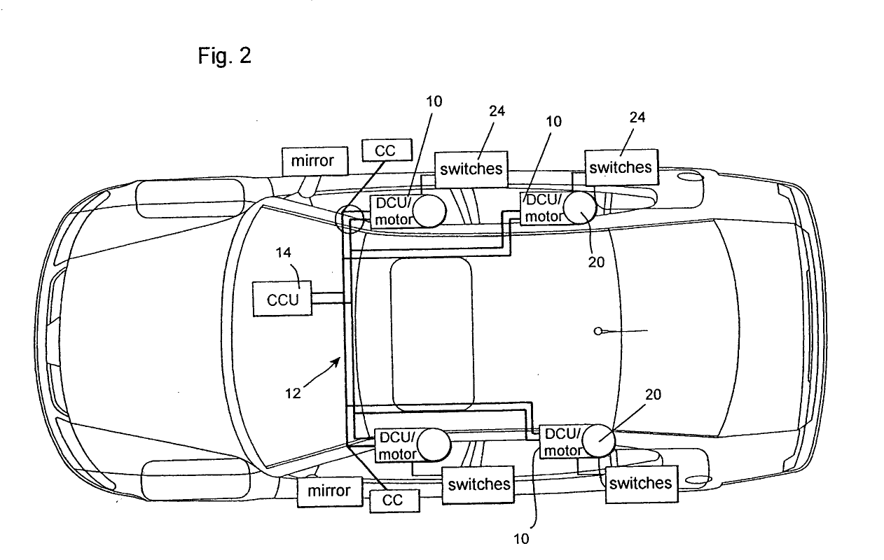

The DCU 10 is connected to a bus system 12 that is preferably a LIN (Local Interconnect Network) bus. Other control units, for example a CCU 14 (Central Control Unit) or DCUs of other vehicle doors (see FIG. 2), communicate with each other via the LIN bus. A LIN protocol is used for this communication. Accordingly, the DCU 10 comprises a first communication module 16 that is adapted to communicate under the LIN protocol, and which is connected with a control module 18 controlling operation of the power window drive, in particular for controlling operation of a motor 20 (see FIG. 2) that drives a displaceable window.

Further, DCU 10 comprises a memory 21, preferably an EEPROM_or a flash controller that is adapted to simulate an EEPROM, which includes data that is unique or specific for a respective vehicle type. For illustration purposes, the following example assumes that memory 21 contains data indicating particulars of an anti-squeeze function of the power window drive, in particular a maximum allowable closing force that is applied to the window. However, memory 21 could contain other specific data. Finally, DCU 10 comprises an input 22 for switches 24 that can be actuated by a vehicle occupant seeking to open or close the window.

It is to be noted that the first communication module 16 is adapted for communication under the LIN protocol only, and is only adapted for normal operation of the DCU 10. “Normal operation” includes opening and closing of the window and certain service functions, such as reading the content of a memory failure that is part of the control module 18, for example.

DCU 10 further includes a second communication module 26 that is also connected to bus system 12, but which is adapted to communicate under a SCI (Serial Connection Interface) protocol only. Second communication module 26 is connected to memory 21 such that its content can be read, modified, deleted or amended in any other way.

Under normal conditions (such as start of the vehicle by switching an ignition on and subsequent operation of the vehicle), communication is made via the first communication module 16 only. The second communication module 26 is inactive and can therefore not be addressed via the bus system 12.

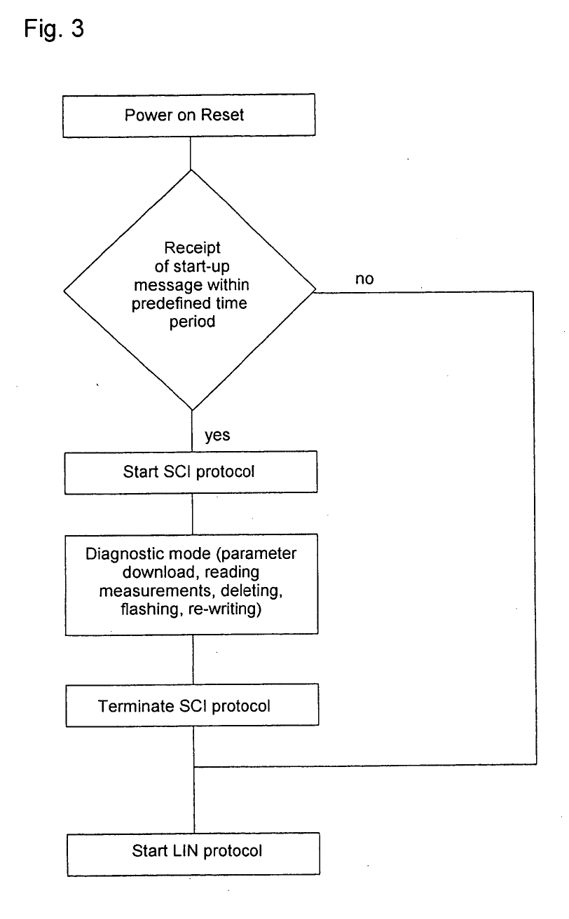

Should a power on reset be performed (see FIG. 3), which involves interrupting power supply from a battery for a time sufficient to ensure that no power remains in associated electrical systems and subsequently re-establishing the power supply, the second communication module 26 “listens” for a predefined time period to determine whether or not a start-up message is received. Such a start-up message is sent from an external diagnostic unit that is temporarily connected to bus system 12. The predefined time period is generally as short as possible and just long enough to allow the diagnostic unit to send the start-up message. A time period of 200 ms or even less should be sufficient, however other time periods could also be used.

Should no start-up message be received within the predefined time period after the power on reset, the second communication module 26 remains inactive, and the first communication module 16 starts operation. DCU 10 remains in the normal operation condition.

Should a start-up message be received within the predefined time period, the second communication module 26 starts the SCI protocol, and the diagnostic unit is permitted access, under the SCI protocol, to the data contained in memory 21 to allow the diagnostic unit to modify data if required. DCU 10 is now in a diagnostic mode.

Access to memory 21 is interrupted and the diagnostic mode is terminated if no diagnostic data is received from the diagnostic unit for a predetermined time period. It could also be provided that the diagnostic unit, in predefined intervals, sends diagnostic session open messages that signify to the second communication module 26 that the diagnostic mode is to be continued. The diagnostic mode would then terminated if no diagnostic session open message is received for a predetermined time period. The diagnostic mode could also be terminated in response to receipt of an explicit diagnostic mode termination message.

After termination of the diagnostic mode, normal operation of the DCU 10 (via the first communication module 16) is enabled, and the LIN protocol is started.

Rather than having the second communication module 26 “listen” for the start-up message after a power on reset, it could be provided that the second communication module 26 is integrated into the first communication module 16 so as to be enabled or started by the first communication module 16 receiving the start-up message. Further, the start-up message could be a predefined condition of the input of the DCU 10 rather than a specific message sent via bus system 12.

It should be understood from the above description that the way and means by which the content of memory 21 is protected against unauthorized and/or unintentional access is applicable to other control units as well, e.g. motor control units, sun roof control units, convertible roof control units, etc.

Although a preferred embodiment of this invention has been disclosed, a worker of ordinary skill in this art would recognize that certain modifications would come within the scope of this invention. For that reason, the following claims should be studied to determine the true scope and content of this invention.

1. A method of operating a control unit comprising the following steps:
performing a power on reset;
entering a diagnostic mode in which a diagnostic communication can be established with a diagnostic unit if the control unit receives a start-up message within a predefined time period after the reset; and
entering a normal mode of operation if no start-up message is received within the predefined time period.

2. The method according to claim 1 including using a first protocol to communicate with the control unit in the normal mode of operation, and using a second protocol different from the first protocol to communicate with the control unit in the diagnostic mode.

3. The method according to claim 2 wherein the first protocol is a local interconnect network (LIN) protocol.

4. The method according to claim 2 wherein the second protocol is a serial connection interface (SCI) protocol.

5. The method according to claim 2 wherein the first protocol is a local interconnect network (LIN) protocol and the second protocol is a serial connection interface (SCI) protocol.

6. The method according to claim 1 including verifying whether or not diagnostic data is continuing to be received after entering the diagnostic mode, and terminating the diagnostic mode when no diagnostic data is received for a predefined time period.

7. The method according to claim 1 including verifying whether or not a diagnostic session open message is being periodically received after entering the diagnostic mode, and terminating the diagnostic mode if no diagnostic session open message is received for a predefined diagnostic time period.

8. The method according to claim 1 including terminating the diagnostic mode after receiving a diagnostic mode termination message.

9. The method according to claim 1 including allowing the diagnostic mode to modify parameters of operation of the control unit.

10. The method according to claim 9 wherein the control unit controls operation of a system associated with a vehicle door and wherein the parameters of operation comprise parameters of an anti-squeeze function.

11. The method according to claim 1 including communicating with the control unit with a bus system.

12. The method according to claim 10 wherein the bus system is a local interconnect network (LIN) bus.

13. The method according to claim 11 including sending the start-up message via the bus system.

14. The method according to claims 1 including defining the start-up message by a specific condition on an input of the control unit.

15. A control unit comprising:
a memory designated to store data unique for a respective vehicle type;
a first communication module allowing communication via a bus system under a first protocol for normal operation of the control unit; and
a second communication module allowing communication via the bus system under a second protocol for accessing the memory.

16. The control unit according to claim 15 wherein the memory is an EEPROM.

17. The control unit according to claim 15 wherein the memory is a flash controller adapted to simulate an EEPROM.

18. The control unit according to claim 15 wherein the control unit is a vehicle door control unit.

19. The control unit according to claim 15 wherein the first protocol comprises a local interconnect network (LIN) protocol and the second protocol comprises a serial connection interface (SCI) protocol.
